(19) United States
(12) Patent Application Publication
(10) Pub. No.: US 2005/0009836 A1
(43) Pub. Date: Jan. 13, 2005

(54) Ophthalmic composition containing quinolones and method of use

(76) Inventors: Paul A. Laskar, Napa, CA (US); Shawn D. Hickok, Napa, CA (US)

Correspondence Address: PERKINS COIE LLP, P.O. BOX 2168, MENLO PARK, CA, 94026, US

(21) Appl. No.: 10/877,280

(22) Filed: Jun. 24, 2004

Related U.S. Application Data
(60) Provisional application No. 60/483,295, filed on Jun. 26, 2003.

(51) Int. Cl. A61K 31/496; A61K 31/4709

(52) U.S. Cl. 514/253.08; 514/312

(57) ABSTRACT

A preservative-free opthalmic composition for treating eye infections is disclosed. The composition contains a quinolone compound in an amount effective as an antibiotic when the composition is placed in the eye. The composition is rendered isoosmotic with polyhydric alcohol. The composition may be utilized to treat ophthalmic conditions by topically applying the composition to the affected tissues.

This patent application claims priority to U.S. Ser. No. 60/483,295 filed Jun. 26, 2003, which is incorporated in its entirety herein by reference.

### FIELD OF THE INVENTION

The present invention relates to ophthalmic compositions containing quinolones and to methods for using such solutions for treating infections and preserving solutions.

### BACKGROUND OF THE INVENTION

Eye infections account for a large proportion of the workload in ophthalmic centers. These infections may be vision- or life-threatening, and prompt treatment of patients with these infections is essential.

A number of antibiotic components, including quinolones, are currently used in ocular application to control, manage or prevent ocular infections (Schwab, I. R., et al. (2003) Ophthalmology 110(3):457-65). For example, a topical ophthalmic composition containing the quinolone ciprofloxacin is marketed by Alcon Laboratories, Inc., and a topical otic composition containing a combination of ciprofloxacin and hydrocortisone is marketed by Alcon Laboratories, Inc. Ofloxacin, norfloxacin and lomefloxacin, levofloxacin, moxifloxacin, gatifloxacin, have also been utilized in ophthalmic antibiotic compositions.

These quinolone antibiotic compositions are generally effective in treating ophthalmic infections, and have distinct advantages over prior ophthalmic antibiotic compositions, particularly those having relatively limited spectrums of antimicrobial activity, such as: neomycin, polymyxin B, gentamicin and tobramycin, which are primarily useful against gram negative pathogens; and bacitracin, gramicidin, and erythromycin, which are primarily active against gram positive pathogens. However, despite the general efficacy of the ophthalmic quinolone therapies currently available, many of these ophthalmic compositions contain preservatives which cause pain, irritation, allergic reactions and/or other harmful side-effects in and around the eye.

U.S. Pat. Nos. 6,492,361 and 6,166,012 disclose preservative-free ophthalmic compositions that include a quinolone component. However, all of the disclosed compositions in this patent which are preservative-free fail either Criteria A or Criteria B or both of the European Pharmacopoeia Efficacy of Antimicrobial Preservation. These compositions, while representing an advance over compositions containing preservatives, do not provide compositions that are capable of passing required efficacy of preservation testing. Thus, these compositions present the possibility of unacceptable microbial contamination over time. The present invention addresses and overcomes these problems.

### SUMMARY OF THE INVENTION

In one aspect, the invention includes an ophthalmic composition consisting essentially of a quinolone compound and an aqueous carrier vehicle. The quinolone compound is levofloxacin, moxifloxacin, gatifloxacin, or ofloxacin, in an amount effective as an antibiotic when the composition is placed in the eye. The aqueous carrier vehicle contains a polyhydric alcohol at a concentration that renders the composition substantially isoosmotic.

The composition is characterized by: (i) having a pH between 6 and 7, (ii) lacking an antimicrobial preservative component other than that possessed by the quinolone compound, (iii) being sufficiently self-preserved to pass the European Pharmacopoeia Efficacy of Antimicrobial Preservation, Criteria B and A, except for A. niger, and (iv) being substantially nontoxic to human corneal keratocytes, as evidenced by the lack of toxicity observed when the composition is added to a cell culture of human keratocytes to a final concentration equivalent to administering a concentration of up to 10%.

The quinolone compound may be levofloxacin, at a concentration between 0.3 and 4.0% w/v, preferably between 1.0 and 3.0% w/v. The polyhydric alcohol may be one or more of the alcohols, glycerin, propylene glycol, polyethylene glycol having an average molecular weight less than 1000 daltons, mannitol or sorbitol. In various embodiments, the polyhydric alcohol includes glycerin at a concentration of about 2.3 v/v percent, propylene glycol, at a concentration of about 2 v/v percent, polyethylene glycol having an average molecular weight between 200 and 1500 daltons and a concentration between about 2 and 8% w/v, mannitol, at a concentration of about 4% w/v, or sorbitol, at a concentration of about 4% w/v.

The composition is used, in another aspect of the invention, in treating an ophthalmic infection, by placing the composition in the affected eye, e.g., in droplet form.

In another aspect, the invention includes an improvement for enhancing the self-preservative property of a self-preserved ophthalmic composition containing a quinolone compound in an aqueous carrier vehicle containing an isoosmotic amount of physiologically acceptable salt, and substantially lacking in any preservative component other than the quinolone compound. The improvement, which includes substituting for the physiologically acceptable salt, a polyhydric alcohol at a concentration that renders the composition substantially isoosmotic, is effective to render the composition sufficiently self-preserved to pass the European Pharmacopoeia Efficacy of Antimicrobial Preservation, Criteria B and A, except for A niger, as well as the corresponding tests in the USP & JP.

In a related aspect, the invention is directed to a method of preserving an aqueous solution of a quinolone compound, for use in treating an ophthalmic infection, without any exogenous antimicrobial preservative component and sufficient to pass the European Pharmacopoeia Efficacy of Antimicrobial Preservation, Criteria B and A, except for A niger. The method involves adding to the solution, an amount of a polyhydric alcohol sufficient to render the solution substantially isoosmotic.

These and other objects and features of the invention will be more fully appreciated when the following detailed description of the invention is read in conjunction with the accompanying figures.

### BRIEF DESCRIPTION OF THE FIGURES

FIGS. 1A-1D show preferred quinolones: levofloxacin (FIG. 1A), moxifloxacin (FIG. 1B,), gatifloxacin (FIG. 1C,) and ofloxacin (FIG. 1D);

FIG. 2 shows the results for the test formulation containing 1.5% levofloxacin plus 2.2% glycerin versus six microorganisms;

FIG. 3 shows A. niger APE testing log reduction values versus time for formulations containing varying levels of levofloxacin;

FIG. 4 shows C. albicans APE testing log reduction values versus time for formulations containing varying levels of levofloxacin;

FIG. 5 shows A. niger APE testing log reduction values versus time for formulations containing 1.5% levofloxacin;

FIG. 6 shows C. albicans APE testing log reduction values versus time for formulations containing 1.5% levofloxacin;

FIG. 7 shows A. niger APE testing log reduction values versus time for formulations containing various excipients;

FIG. 8 shows C. albicans APE testing log reduction values versus time for formulations containing various excipients; and

FIGS. 9-12 show human keratocyte bioassay results from various compositions prepared according to certain embodiments of the invention.

### DETAILED DESCRIPTION OF THE INVENTION

I. Definitions

Unless otherwise indicated, all technical and scientific terms used herein have the same meaning as they would to one skilled in the art of the present invention. It is to be understood that this invention is not limited to the particular methodology, protocols, and reagents described, as these may vary.

The term “ophthalmic composition” refers to a substance which can be used for the treatment or prevention of eye diseases or disorders, such as infections.

The term “ophthalmic infections” refers to inflammation of or around the eye, e.g. conjunctiva (conjunctivitis) and cornea (conjunctivitis), caused by microorganisms such as staphylococci, streptococci and/or enterococci.

As used herein, the term “ophthalmically acceptable” refers to a material which, at the concentration or amount in question, is compatible with ocular tissue. Thus, an ophthalmically acceptable component does not cause significant or undue detrimental effects when brought into contact with ocular tissue.

By “quinolone” is meant an antibiotic having a naphthyridine nucleus or quinoline nucleus with different side chains as understood in the art (DaSilva, A D, et al. (2003) Biological activity and synthetic methodologies for the preparation of fluoroquinolones, a class of potent antibacterial agents, Curr Med Chem 10(1):21-39; and Yao, J. D. C. et al. (1995) In: Murray, P. R. et al., eds. Manual of Clinical Microbiology, ASM Press, Washington, D.C. pp. 1288-1290). Quinolones include, but are not limited to, oxolinic acid, cinoxacin, flumequine, miloxacin, rosoxacin, pipemidic acid, norfloxacin, enoxacin, moxifloxacin, gatifloxacin, ciprofloxacin, ofloxacin, lomefloxacin, temafloxacin, fleroxacin, pefloxacin, amifloxacin, sparfloxacin, levofloxacin, clinafloxacin and nalidixic acid.

As used herein, “in amounts effective”, “an amount effective”, or “an effective amount”, refer to the amount of antibiotic administered which is effective to reduce the rate of microbial infection, microbial growth and/or the symptoms associated with microbial infection.

“Treatment of” or “treating” a subject is any type of intervention provided as a means to alter the natural course of the subject. Treatment includes, but is not limited to, administration of e.g., a pharmaceutical composition, and may be performed either prophylactically, or subsequent to the initiation of a pathologic event or contact with an etiologic agent. The related term “improved therapeutic outcome” relative to a patient diagnosed as infected with a particular microbe, refers to a slowing or diminution in the growth of the microbe, or detectable symptoms associated with infection by that particular microbe.

The term “lacking an antimicrobial preservative component” as used herein means present in quantities that have less than a material effect on, or confer less than a material advantage to, the pharmaceutical composition.

The term “toxicity” refers to any adverse and/or side effect of a composition on the metabolism of a cell or an ophthalmic tissue. The amount of toxicity associated with a composition may vary with several conditions including, but not limited to, the amount of composition present, the formulation of the drug and the environmental conditions of the affected cell, organ and/or subject.

The term “preservative efficacy” or “preservative effectiveness”, as used herein, means that the composition satisfies the preservative standards as defined in the protocols European Pharmacopoeia Efficacy of Antimicrobial Preservation, Criteria B and A, except for A. niger (PhEur, 4^{th} ed, 4.4).

European Pharmacopoeia Efficacy of Antimicrobial Preservation, Criteria B and A, is met for a solution composition if (a) the concentrations of viable bacteria are reduced to not more than 0.1% of the initial concentrations by the fourteenth day; (b) the concentrations of viable yeasts and molds remain at or below the initial concentrations during the first 14 days; and (c) the concentration of each test microorganism remains at or below these designated levels during the remainder of the 28-day test period (PhEur, 4^{th} ed, 4.4). Differing criteria for A and B include:

Bacteria

| Category | Time | Log reduction |
|---|---|---|
| A | 24 h | 3 |
| B | 7 d | 3 |

And No Recovery at 28d

Yeasts & Molds:

| Category | Time | Log reduction |
|---|---|---|
| A | 7 d | 2 |
| B | 14 d | 1 |

And No Increase at 28d.

“Except A. niger,” in the context of European Pharmacopoeia Efficacy of Antimicrobial Preservation, Criteria B and A, means that the preservative criteria A of the European Pharmacopoeia Efficacy of Antimicrobial Preservation is not met for the mold, A niger.

The term “subject” refers to a mammal, preferably a human.

II. Ophthalmic Composition

The invention provides, in one aspect, an ophthalmic composition. It has been discovered that an ophthalmic, preservative-free quinolone composition having specific components, as described below, is effective to reduce the risk of formation of microbial infections and/or to reduce the severity of inflammation or pain caused by such infections. It should be appreciated that although the composition lacks an antimicrobial preservative component other than that possessed by the quinolone compound, the composition is sufficiently self-preserved to pass Criteria B and A, except for A niger, of the European Pharmacopoeia Efficacy of Antimicrobial Preservation. Considered below are the components of the composition of the invention.

A. Quinolone Compound

The antibiotics utilized in the composition of the present invention are classified as quinolones. These compositions include an antibiotically effective amount of the quinolone component. Such amounts may vary over a relatively broad range depending, for example, on the specific form of the composition being used, the specific quinolone component being used, the specific application for the composition, the frequency of use of the composition and the like factors. In one embodiment, the present compositions include a quinolone component in an amount in a range of about 0.3% w/v or less to about 4% w/v or more. Preferably, the quinolone component is in an amount in the range of about 1.0% w/v to about 3.0% w/v.

The quinolone compound is present in an amount effective as an antibiotic when the composition is placed in a mammalian eye, preferably a human eye. An effective amount provides one or more benefits to the subject treated, such as prevention, control or management of microbial infections, and/or reduction in inflammation and/or pain. In one embodiment, the quinolone compound is levofloxacin at a concentration between about 0.1 and about 6.0% w/v, preferably between about 0.3 and 4.0% w/v, and more preferably between about 1.0 and about 3.0% w/v.

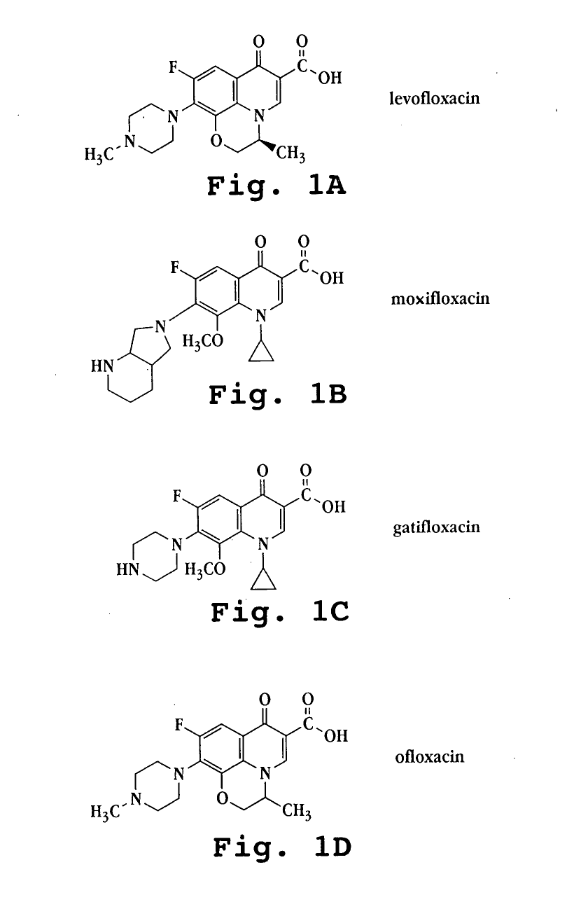

Preferred fluorinated quinolones are second generation quinolones, such as ofloxacin, ciprofloxacin, grepafloxacin, and the next generation quinolones, such as levofloxacin, moxifloxacin and gatifloxacin. These quinolones offer advantages over earlier fluoroquinolones, possessing a broader-spectrum bactericidal activity and excellent bioavailability. In addition, they do not appear to promote the development of resistant strains. See, e.g., Higgins P. G., et al. (2003) Fluoroquinolones: structure and target sites, Curr Drug Targets 4(2): 181-90; Blondeau, J. M. (1999) A review of the comparative in-vitro activities of 12 antimicrobial agents, with a focus on five new respiratory quinolones, J. of Antimicrobial Chemotherapy, 43, Suppl. B. 1-11; Tillotson, G. S. (1996) Quinolones: structure-activity relationships and future predictions, J. of Medical Microbiology, 44, 320-4; Gootz, T. D. and Brighty, K. E. (1996) Fluoroquinolone antibacterials: SAR mechanism of action, resistance, and clinical aspects, Medicinal Research Reviews 16, 433-86; and Wentland, M. P. (1990) Structure-activity relationships of fluoroquinolones, In the New Generation of Quinolones, (Siporin, C., Heifetz, C. L. & Domagala, J. M., Eds), pp. 1-43, Marcel Dekker, New York; each of which is incorporated herein by reference. Particularly preferred quinolones are levofloxacin (FIG. 1A), moxifloxacin (FIG. 1B), gatifloxacin (FIG. 1C) and ofloxacin (FIG. 1D).

Details regarding the structure, preparation, and physical properties of moxifloxacin and related compounds are provided in U.S. Pat. No. 5,607,942, which is incorporated by reference herein. Details regarding the structure, preparation, and physical properties of gatifloxacin and related compounds are provided in U.S. Pat. No. 4,980,470, which is incorporated by reference herein. Additional methods for the preparation of quinolones may be found in DaSilva, A D, et al. (2003) Biological activity and synthetic methodologies for the preparation of fluoroquinolones, a class of potent antibacterial agents, Curr Med Chem 10(1):21-39, which is incorporated herein by reference.

B. Polyhydric Alcohol

The ophthalmic composition of the invention contains an aqueous carrier vehicle for administration. Preferably, the carrier is a polyhydric alcohol that is at a concentration that renders the composition substantially isoosmotic with physiological fluids. In one embodiment, the composition is adjusted to have an osmolarity of between about 230 to about 450 mOsm, preferably 260-320 mOsm.

Suitable polyhydric alcohols include ethylene glycol, propylene glycol-(1,2) and -(1,3), butylene glycol-(1,4) and -(2,3), hexanediol-(1,6), octanediol-(1,8), neopentyl glycol, 1,4-bis(hydroxymethyl)cyclohexane, 2-methyl-1,3-propanediol, glycerin, trimethylolethane, hexanetriol-(1,2,6), butanetriol-(1,2,4), quinol, methyl glucoside, triethyleneglycol, tetraethylene glycol and higher polyethylene glycols of molecular weight between about 100 and about 1000, dipropylene glycol and higher polybutylene glycols, diethylene glycol, glycerin, pentaerythritol, trimethylolpropane, sorbitol, mannitol, dibutylene glycol and higher polybutylene glycols. Preferably, the polyhydric alcohol is glycerol, propylene glycol, polyethylene glycol having an average molecular weight less than 1000 daltons, mannitol and/or sorbitol.

In one embodiment, the polyhydric alcohol includes glycerin at a concentration of about 2.3 v/v %. In another embodiment, the polyhydric alcohol includes propylene glycol at a concentration of about 2 v/v %.

An exemplary polyhydric alcohol includes polyethylene glycol having an average molecular weight between 200 and 1500 daltons and a concentration between about 2 and 8% w/v. In one embodiment, the polyhydric alcohol includes mannitol, at a concentration of about 4% w/v. In another embodiment, the polyhydric alcohol includes sorbitol, at a concentration of about 4% w/v.

Preferably, the quinolone compound is levofloxacin, present at a concentration between 1.0 and 2.0% w/v, and the polyhydric alcohol is glycerin, present at a concentration of between 2 and 2.5 v/v%.

C. pH

The ophthalmic composition of the present invention is advantageously adjusted to a pH range generally employed for eye solutions, which, in one embodiment is from about 3 to 8, preferably from about 4 to 7.5, and more preferably between about 6 and 7. For adjusting the pH, various ophthalmically acceptable acids and/or bases may be used.

Acids optionally useful in the present compositions include boric acid, hydrochloric acid, acetic acid, other acids which are ophthalmically acceptable in the concentrations used, and the like. Bases which may be included in the present compositions include, but are not limited to, sodium and/or potassium hydroxides, other alkali and/or alkaline earth metal hydroxides, organic bases, other bases which are ophthalmically acceptable in the concentrations used, and the like.

D. Self-Preservation

As noted above, the ophthalmic composition of the invention lacks an antimicrobial preservative component other than that possessed by the quinolone compound, but is still sufficiently self-preserved. The antimicrobial effectiveness of the ophthalmic composition of the present invention may be determined using an organism challenge test according to the methods described in the United States Pharmacopia (USP), European Pharmacopoeia (Ph. Eur.), and/or Japanese Pharmacopia (JP), each of which is incorporated herein by reference.

Generally, these tests involve inoculating samples with known levels of gram-positive (e.g., Staphylococcus aureus) and gram-negative (e.g., Pseudomonas aeruginosa and Escherichia coli) vegetative bacteria, yeast (e.g., Candida albicans) and mold (e.g., Aspergillus niger). The inoculated samples are tested at specified intervals to determine if the antimicrobial preservative system is capable of killing or inhibiting the propagation of organisms purposely introduced into the formulation. The rate or level of antimicrobial activity determines compliance with the USP, Ph. Eur. and/or JP preservative efficacy standard for ophthalmic preparations. See, e.g., U.S. Pat. Nos. 6,181,963 and 6,004,968, which are incorporated by reference herein.

In one embodiment, the composition is sufficiently self-preserved to pass the European Pharmacopoeia Efficacy of Antimicrobial Preservation Criteria B and A, except for A. niger. Preferably, the composition is sufficiently self-preserved to pass the European Pharmacopoeia Efficacy of Antimicrobial Preservation Criteria B and A, except for A. niger, as well as the corresponding tests in the USP and/or JP.

Exemplary formulations containing preservative-free compositions that are capable of passing at least the USP Antimicrobial Effectiveness testing are given in Examples B, D and E below.

E. Toxicity

A variety of test models and protocols may be used in the invention as in vitro screens for assessing ocular toxicity or irritation. See, e.g., Booman, K. A. et al. (1988) In vitro methods for estimating eye irritancy of cleaning products, Phase I: Preliminary assessment, J. Toxicol. Cut & Ocular Toxicol. 7:173-185, which is incorporated herein by reference. Cell cultures used in conjunction with quantifiable, objective endpoints for assessing cytotoxicity have shown good correlation to in vivo data sets. Bruner, L. H, et al. (1991) Evaluation of seven in vitro alternatives for ocular safety testing, Fund. Appl. Toxicol. 17:136-149, which is incorporated herein by reference. A preferred method for assessment involves adding the composition to a cell culture of human corneal keratocytes to a final concentration corresponding to an ocular composition having a concentration of about 5% to 15%, preferably about 10%, and observing substantial lack of toxicity. An exemplary human keratocyte bioassay method for assessing toxicity is described in Example F, below.

Additional methods for testing toxicity contemplated by the invention include the in vitro cornea equivalent model as disclosed in U.S. Pat. No. 5,827,641, which is incorporated herein by reference. Toxicity in vivo is primarily manifested by allergic reactions to excipients or active ingredients in topical antibacterial preparations (Robert P. Y. and Adenis J. P. (2001) Comparative review of topical ophthalmic antibacterial preparation, Drugs 61(2):175-185).

III. Method of the Invention

The invention provides, in one aspect, a method of treating an ophthalmic condition in a subject. The method includes administering one or more of the foregoing compositions to a subject so as to provide one or more of the desired benefits. Such benefits include prevention, control or management of ocular microbial infections and/or the symptoms associated therewith, e.g. inflammation and/or pain.

A. Administering the Composition

The self-preserved formulations, as described in Section II above, are useful in storing and administering therapeutic ophthalmic compositions according to the methods described herein. Preferably, the method includes topical administration of the composition in the subject's eye. Such administration may include, but is not limited to, topical application to the eye, installation into the eye, placing an insert into the cul-de-sac (space) between the eyeball and the eyelid and the like. Other conventional methods of administering ophthalmic compositions to the eye may be employed and are well known to those of skill in the art.

The dosage level of the foregoing compositions may depend on a number of factors, including, but not limited to, the particular application involved, the particular quinolone component employed, the concentration of the quinolone component in the composition, the severity of the infection and/or the subject's response to the treatment. Such dosage can be easily determined by routine and well known techniques to achieve the desired result in the subject being treated. Physicians may adjust the number of doses per day, the time between doses, and the length of treatment with the composition. Techniques for administration of pharmaceutical ophthalmic compositions may be found in Remington's Pharmaceutical Sciences (1995) 19th Ed., Williams & Wilkins, which is incorporated by reference herein, and are well known to those skilled in the art.

Ophthalmic conditions contemplated for treatment include conjunctivitis, keratitis, blepharitis, dacyrocystitis, hordeolum, and corneal ulcers. Bacterial conjunctivitis is the most common form of infectious conjunctivitis and bacterial keratitis accounts for 65-90% of all bacterial corneal infections. Additional conditions contemplated for treatment include infection or a risk of infection resulting from trauma to ophthalmic tissue. Trauma associated with contamination by vegetative material, contact lens wear and long term corticosteroid use are common risk factors. The method may also include prophylactic treatment in connection with various ophthalmic surgical procedures that create a risk of infection. The following conditions may also be treated with the compositions of the invention: preseptal cellulitis (periorbital cellulitis), which is an infectious inflammation of the tissues anterior to the orbital septum, more often encountered in children with upper respiratory infections; orbital cellulitis, which is an infectious inflammatory process involving the orbital tissues posterior to the orbital septum; and mucormycosis, which is a fulminant oportunisitic fungal infection caused by fungi of the class Zygomycetes. The infection typically begins in the paranasal sinuses and spreads to the orbit. The large, nonseptate hyphae cause vascular occlusion. This causes ischemia and infarction of tissue.

Efficacy of the method of treatment may be conveniently measured by any of the standard indicators of reduced or eliminated ophthalmic infection. The present methods may be curative and/or preventative when applied. The method may be utilized presurgically and/or post-traumatically. Thus, the method may be applied prior to a microbial infection, or before inflammation and/or pain is apparent, or following such infection. Use of the method is effective to reduce the risk of the formation of such infections.

In general, synergistic and optimized doses of the present invention are established by determining the concentrations of components that provide the desired effect in a test system, such as the cell systems described herein, or in other suitable in vitro or in vivo model systems. Based on such data, efficacious doses for administration to human (or other animal) subjects can be determined, for example, based on known or routinely ascertainable pharmacokinetics of the specific compound in humans (See, e.g., Benet, L. Z., et al. (1996), in Goodman and Gilman's The Pharmacological Basis of Therapeutics, Ninth Ed., Hardman, J. G., et al., eds., McGraw-Hill, San Francisco; Wagner, J. G., (1993) Pharmacokinetics for the Pharmaceutical Scientist. Technomic, Inc., Lancaster, Pa.; Rowland, M., and Tozer, T. N., (1995) Clinical Pharmacokinetics: Concepts and Applications, 3rd ed., Lea & Febiger, Philadelphia).

B. Solution Preservation

The invention includes, in another aspect, a method of preserving an aqueous solution of a quinolone compound without any exogenous antimicrobial preservative component. The preserved solution is preferably used for treating an ophthalmic infection, as discussed above.

The method includes adding to the solution an amount of a polyhydric alcohol sufficient to render the solution substantially isoosmotic. In a preferred embodiment, the quinolone compound is selected from the group consisting of levofloxacin, moxifloxacin, gatifloxacin and ofloxacin, and said polyhydric alcohol is selected from the group consisting of glycerin, propylene glycol, polyethylene glycol having an average molecular weight less than 1000 daltons, mannitol and sorbitol.

From the foregoing, it can be seen how various objects and features of the invention are met.

IV. Examples

The following examples further illustrate the invention described herein and are in no way intended to limit the scope of the invention.

A. Presentation of USP Antimicrobial Effectiveness (APE) Test Results

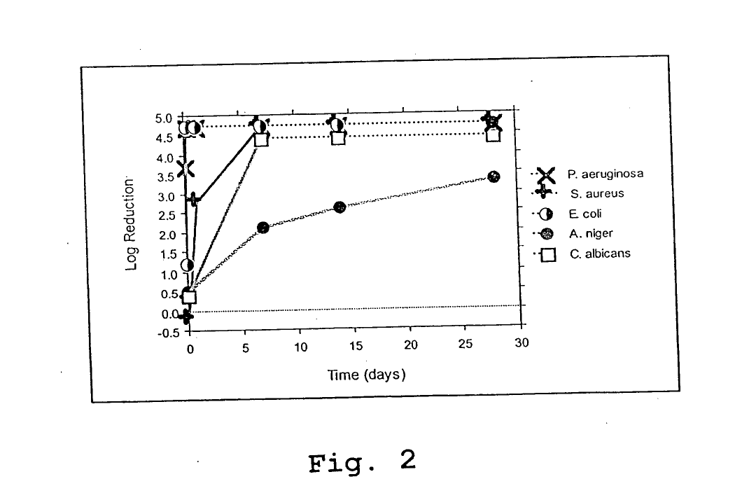

The APE testing results are plotted and analyzed in FIGS. 2-8 as “Log Reduction” versus time, where:

Log Reduction=logarithm₁₀{Control (cfu/mL)}−logarithm₁₀{Test Sample (cfu/mL)}

Effectiveness in the APE test results is indicated by a rapid increase of “Log Reduction” values to 4 or 5 as a function of time.

Formulation development studies evaluated sensitivity of A. niger, C. albicans, S. aureus and P. aeruginosa. Formulations were extremely effective against these microbes.

B. APE Results for Aged Formulation Against Five Standard Test Organisms

TABLE 1
Composition of formulation shown in FIG. 2

| Ingredient | Formulation |
|---|---|
| Levofloxacin (%) | 1.5 |
| Glycerin (%) | 2.2 |
| BAK (%) | 0 |
| NaCl (%) | 0 |
| pH | 6.5 |

C. Formulations Containing Varying Levels of Levofloxacin without Glycerin

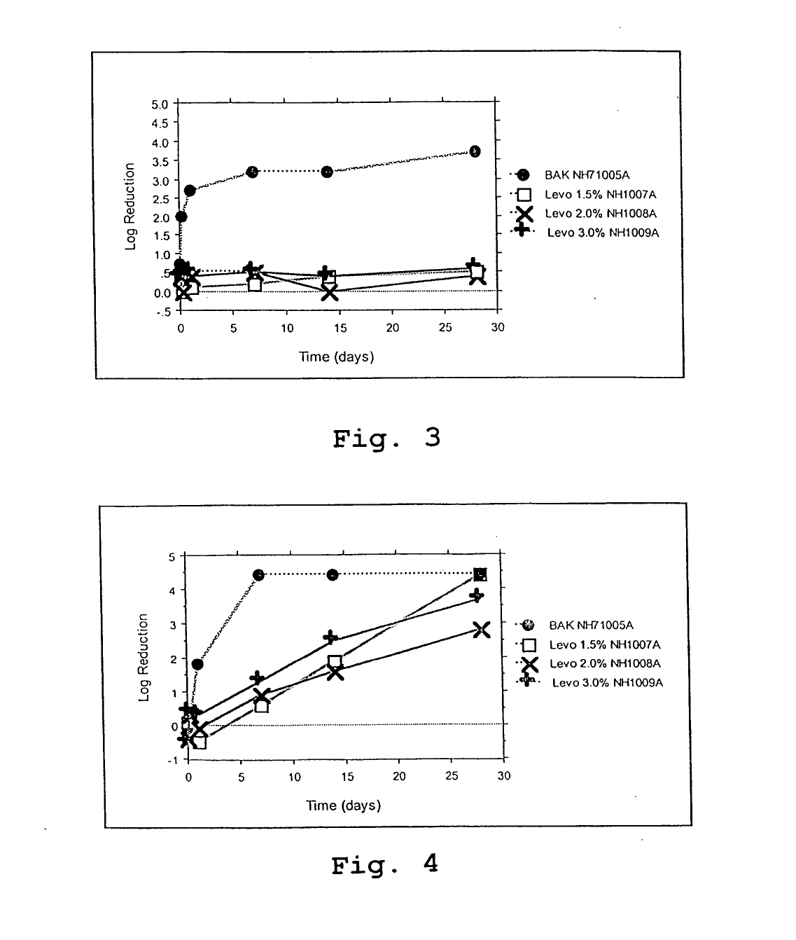

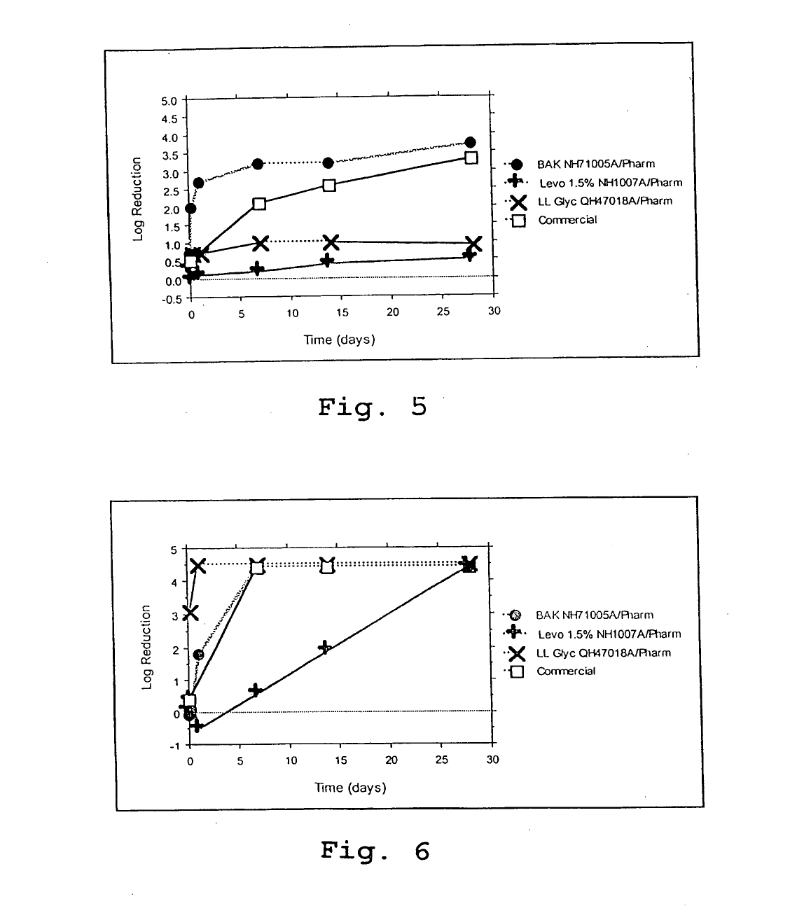

TABLE 2
Composition of formulations shown in FIGS. 3 and 4

| | Formulation # “Name” | | | |
|---|---|---|---|---|
| | “BAK | | | |
| Ingredient | Control” | “Levo 1.5” | “Levo 2.0” | “Levo 3.0” |
| Levofloxacin (%) | 0 | 1.5 | 2.0 | 3.0 |
| Glycerin (%) | 0 | 0 | 0 | 0 |
| BAK (%) | 0.0050 | 0 | 0 | 0 |
| NaCl (%) | 0.90 | 0.81 | 0.78 | 0.72 |
| PH | 6.6 | 6.6 | 6.6 | 6.6 |

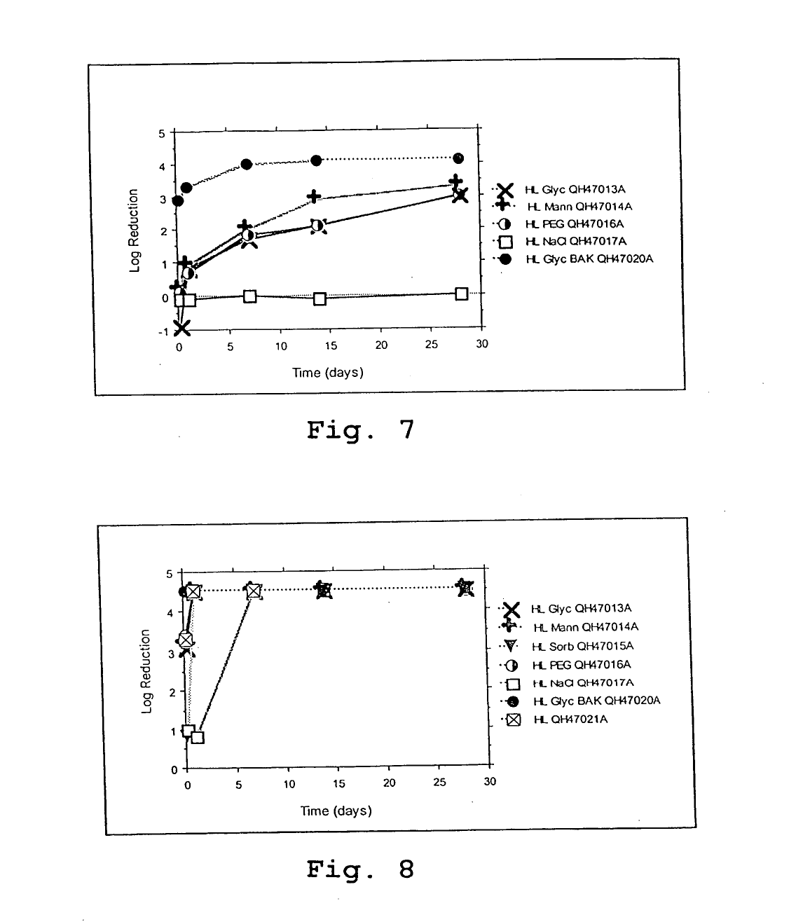

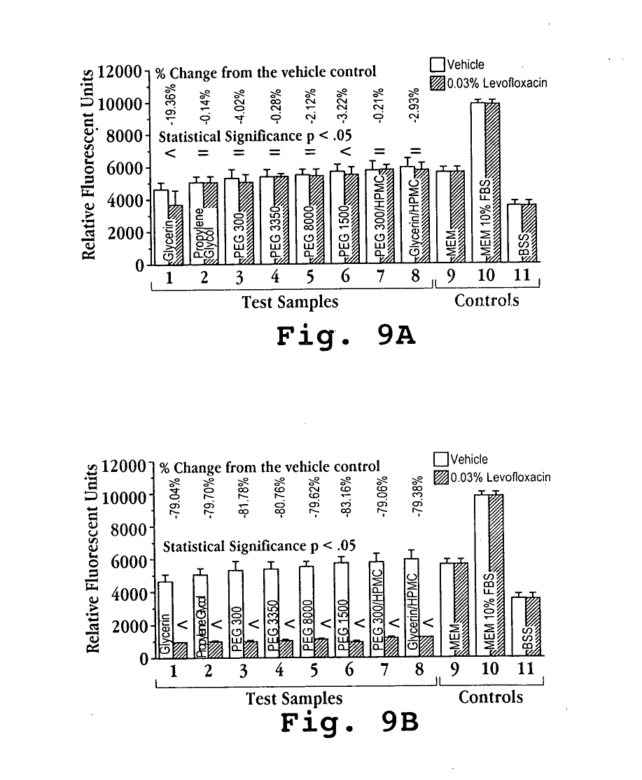

A. niger and C. albicans APE testing values versus time indicate that the formulations containing varying levels of levofloxacin are not as effective as the BAK positive control sample. Furthermore, increasing levofloxacin levels, as shown in Table 2 and FIGS. 5 and 6, does not appear to significantly improve APE performance of the formulations.

D. Formulations Containing Levofloxacin Plus Varying Levels of Glycerin

TABLE 3
Composition of formulations shown in FIGS. 3 and 4

| | Formulation # “Name” | | | |
|---|---|---|---|---|
| | | | “Levo 1.5 + | “Levo 1.5 + |
| | “BAK | “Levo 1.5 + | 2.3% | 2.2% |
| Ingredient | Control” | Saline” | Glycerin” | Glycerin” |
| Levofloxacin (%) | 0 | 1.5 | 1.5 | 1.5 |
| Glycerin (%) | 0 | 0 | 2.3 | 2.2 |
| BAK (%) | 0.0050 | 0 | 0 | 0 |
| NaCl (%) | 0.90 | 0.81 | 0 | 0 |
| pH | 6.6 | 6.6 | 6.65 | 6.5 |

APE results for formulations containing 1.5% levofloxacin plus or minus 2.2% glycerin versus a BAK control sample (see Table 3) against A. niger and C. albicans are shown in FIGS. 5 and 6. The results shown in FIGS. 5 and 6 indicate that formulations containing levofloxacin and glycerin give enhanced APE performance versus formulations containing levofloxacin alone.

E. Formulations Containing Levofloxacin Plus Varying Levels of Other Charged and Uncharged Excipients

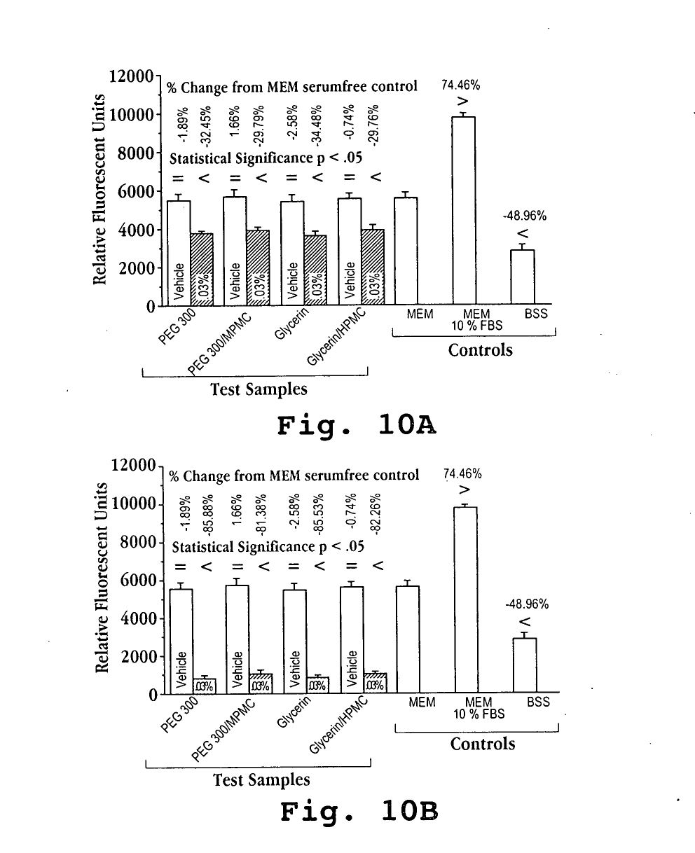

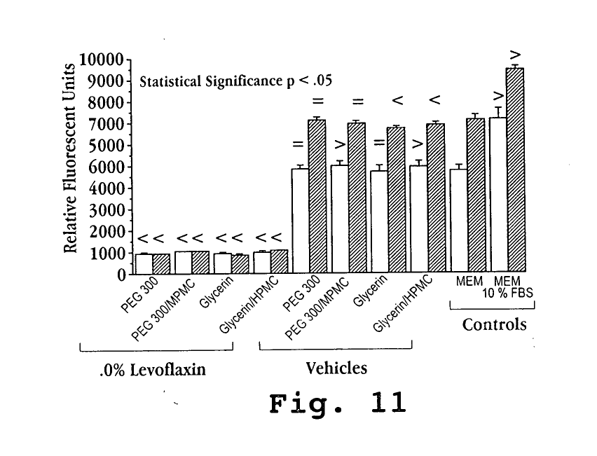

TABLE 4
Composition of formulations shown in FIGS. 7 and 8

| | Formulation # “Name” | | | | | | |
|---|---|---|---|---|---|---|---|
| | | | | “Levo | | “Levo 3.1 + | |
| | “Levo 3.1 + | “Levo 3.1 + | “Levo 3.1 + | 3.1 + PEG- | “Levo | Glycerin + | “Levo |
| Ingredient | Glycerin” | Mannitol” | Sorbitol” | 300” | 3.1 + NaCl” | BAK” | 3.1” |
| Levofloxacin | 3.1 | 3.1 | 3.1 | 3.1 | 3.1 | 3.1 | 3.1 |
| (%) | | | | | | | |
| Glycerin (%) | 2.1 | — | — | — | — | 2.1 | — |
| Mannitol (%) | — | 4.0 | — | — | — | — | — |
| Sorbitol (%) | — | — | 4.0 | — | — | — | — |
| PEG-300 (%) | — | — | — | 5.1 | — | — | — |
| NaCl (%) | — | — | — | — | 0.73 | — | — |
| BAK (%) | — | — | — | — | — | 0.001 | — |
| pH | 6.65 | 6.65 | 6.65 | 6.65 | 6.65 | 6.65 | 6.65 |

Other water soluble non-charged molecules show a similar APE increase versus levofloxacin alone formulations as that found for glycerin above. The results for formulations containing 3.1% levofloxacin plus several added excipients are shown in Table 4 and FIGS. 7 and 8. Sodium chloride 0.73% is singular ineffective in this role while 4.0% mannitol, 4.0% sorbitol or 5.1% PEG-300 mimicked the results for 2.1% glycerin.

F. Human Keratocyte Bioassay

Human Corneal Endothelial Cell and Keratocyte Cultures

Ninety-six well tissue culture plates were seeded with third passage human corneal keratocyte (HCK) and endothelial cells (HCE) at 1×10³ cells/well in a final volume of 200 μl of CSM (chondroitin sulfate medium; Insight Biomed, Inc., Minneapolis, Minn., USA) medium supplemented with 10% fetal bovine serum (Hyclone, Logan, Utah, USA). Cells were maintained in a humidified incubator at 35.5° C. in a 95% air: 5% CO₂ atmosphere. After 4 days of incubation in CSM medium, supplemented with 10% FBS, the medium was removed.

The cells were rinsed one time and incubated with the appropriate test or control solutions (levofloxacin and ofloxacin were obtained from Daiichi Pharmaceutical Company Ltd., Japan, and ciprofloxacin, moxifloxacin and gatifloxacin were purchased from LKT Laboratories, Inc., MN) for 15 minutes, 30 minutes, 1 hour, and 4 hours. Test solutions were applied at concentrations of 10 ng/mL, 100 ng/mL, 1 μg/mL, 10 μg/mL, 100 μg/mL, and 1 mg/mL. Designated wells were then rinsed twice with 200 μl of serum-free minimal essential medium (MEM, Sigma-Aldrich, St. Louis, Mo., USA) and incubated with 200 μl of fresh CSM medium, supplemented with 10% FBS for the remainder of the 72 hours. After 72 hours, each well was rinsed two times with 200 μl of commercial BSS (balanced salt solution, Cytosol Laboratories, Inc., Braintree, Mass., USA).

Calcein AM Fluorescent Quantitative Bioassay

Live cells are distinguished by the presence of ubiquitous intracellular esterase activity, determined by the enzymatic conversion of the virtually non-fluorescent cell permeant calcein AM to the intensely fluorescent calcein. The polyanionic calcein is well retained within live cells, producing an intense uniform green (530 nm) fluorescence in live cells. This can be expressed as: Calcein AM (Non-Fluorescent)+Esterases=Calcein (Fluorescent Product)

After exposure to test and control materials, cells were incubated with 100 μl/well of 2 μM Calcein AM solution (Molecular Probes, Inc. Eugene, Oreg., USA) and immediately read on a Millipore CytoFluor™ 2,300 Fluorescence Measurement System (Applied Biosystems, Foster City, Calif., USA). A 485/20 nm excitation wavelength and a 530/25 nm emission wavelength filter set (sensitivity 5) were used to measure the fluorescent product. A Wilcoxen Signed-Rank Test was used to evaluate statistical significance (p<0.05) between the test and control groups. This study was conducted under federal Good Laboratory Practices standards at Insight Biomed, Inc. Minneapolis, Minn., USA.

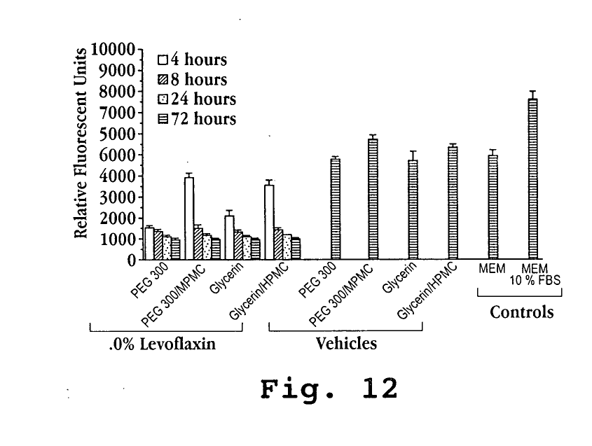

Analysis of Human Keratocyte Bioassay test results, as shown in FIGS. 9-12 indicates that all of the formulations based on organic uncharged excipients give comparable human keratocye biocompatibility.

Although the invention has been described with respect to particular embodiments, it will be apparent to those skilled in the art that various changes and modifications can be made without departing from the invention.

1. An ophthalmic composition consisting essentially of:
(a) a quinolone compound selected from the group consisting of levofloxacin, moxifloxacin, gatifloxacin, and ofloxacin, in an amount effective as an antibiotic when the composition is placed in the eye, and
(b) an aqueous carrier vehicle containing a polyhydric alcohol at a concentration that renders the composition substantially isoosmotic,
said composition being characterized by:
(i) having a pH between 6 and 7,
(ii) lacking an antimicrobial preservative component other than that possessed by the quinolone compound,
(iii) being sufficiently self-preserved to pass the European Pharmacopoeia Efficacy of Antimicrobial Preservation, Criteria B and A, except for A. niger, (and)
(iv) being substantially nontoxic to human corneal keratocytes, as evidenced by the lack of toxicity observed when the composition is added to a cell culture of human keratocytes to a final concentration of 10%.

2. The composition of claim 1, wherein the quinolone compound is levofloxacin, at a concentration between 0.3 and 4.0% w/v.

3. The composition of claim 2, wherein levofloxacin is present at a concentration between 1.0 and 3.0% w/v.

4. The ophthalmic composition of claim 1, wherein the polyhydric alcohol includes one or more alcohols selected from the group consisting of glycerin, propylene glycol, polyethylene glycol having an average molecular weight less than 1000 daltons, mannitol and sorbitol.

5. The composition of claim 4, wherein the polyhydric alcohol includes glycerin at a concentration of about 2.3 v/v percent.

6. The composition of claim 4, wherein the polyhydric alcohol includes propylene glycol, at a concentration of about 2% v/v.

7. The composition of claim 4, wherein the polyhydric alcohol includes polyethylene glycol having an average molecular weight between 200 and 1500 daltons and a concentration between about 2 and 8% w/v.

8. The composition of claim 4, wherein the polyhydric alcohol includes mannitol, at a concentration of about 4% w/v.

9. The composition of claim 4, wherein the polyhydric alcohol includes sorbitol, at a concentration of about 4% w/v.

10. The composition of claim 4, wherein the quinolone compound is levofloxacin, present at a concentration between 1.0 and 2.0% w/v, and the polyhydric alcohol is glycerin, present at a concentration of between 2 and 2.5% v/v.

11. In a self-preserved ophthalmic composition containing a quinolone compound in an aqueous carrier vehicle containing an isoosmotic amount of physioloogically acceptical salt, and substantially lacking in any preservative component other than the quinolone compound, an improvement for enhancing the self-preservative property of the,composition such that the composition is sufficiently self-preserved to pass the European Pharmacopoeia Efficacy of Antimicrobial Preservation, Criteria B and A, except for
A niger, as well as the corresponding tests in the USP & JP, said improvement comprising substituting for the physiologically acceptable salt, a polyhydric alcohol at a concentration that renders the composition substantially isoosmotic.

12. The improvement of claim 11, wherein said quinolone compound is selected from the group consisting of levofloxacin, moxifloxacin, gatifloxacin, and ofloxacin, and said polyhydric alcohol is selected from the group consisting of glycerin, propylene glycol, polyethylene glycol having an average molecular weight less than 1000 daltons, mannitol and sorbitol.

13. A method for treating an ophthalmic infection in a subject, comprising
placing in the subject's eye, a composition consisting essentially of:
(i) a quinolone compound selected from the group consisting of levofloxacin, moxifloxacin, gatifloxacin, and ofloxacin, in an amount effective as an antibiotic when the composition is placed in the eye, and
(ii) an aqueous carrier vehicle containing a polyhydric alcohol at a concentration that renders the composition substantially isoosmotic,
said composition having a pH between 6 and 7, lacking an antimicrobial preservative component other than that possessed by the quinolone compound, and being sufficiently self-preserved to pass the European Pharmacopoeia Efficacy of Antimicrobial Preservation, Criteria B and A, except for A niger.

14. The method of claim 13, wherein the polyhydric alcohol includes one or more alcohols selected from the group consisting of glycerin, propylene glycol, polyethylene glycol having an average molecular weight less than 1000 daltons, mannitol and sorbitol.

15. The method of claim 13, wherein the quinolone compound is levofloxacin, at a concentration beween 0.3 and 4.0% w/v.

16. The method of claim 15, wherein levofloxacin is present at a concentration between 1.0 and 3.0% w/v.

17. The method of claim 16, wherein and the polyhydric alcohol is glycerin, present at a concentration of between 2 and 2.5 v/v %.

18. A method of preserving an aqueous solution of a quinolone compound, for use in treating an ophthalmic infection, without any exogenous antimicrobial preservative component and sufficient to pass the European Pharmacopoeia Efficacy of Antimicrobial Preservation, Criteria B and A, except for A niger, comprising
adding to the solution, an amount of a polyhydric alcohol sufficient to render the solution substantially isoosmotic.

19. The method of claim 18, wherein said quinolone compound is selected from the group consisting of levofloxacin, moxifloxacin, gatifloxacin and ofloxacin, and said polyhydric alcohol is selected from the group consisting of glycerin, propylene glycol, polyethylene glycol having an average molecular weight less than 1000 daltons, mannitol and sorbitol.
